(19) United States
(12) Patent Application Publication
(10) Pub. No.: US 2019/0231802 A1
(43) Pub. Date: Aug. 1, 2019

(54) CHEMOTHERAPEUTIC-RELEASING IMPLANTABLE STICK, METHODS OF MANUFACTURE AND PRECURSOR MATERIALS

(71) Applicant: The Johns Hopkins University, Baltimore, MD (US)

(72) Inventors: Gregory Riggins, Baltimore, MD (US); Renyuan Bai, Baltimore, MD (US); Verena Staedtke, Baltimore, MD (US)

(73) Assignee: The Johns Hopkins University, Baltimore, MD (US)

(21) Appl. No.: 16/342,818

(22) PCT Filed: Oct. 18, 2017
(86) PCT No.: PCT/US2017/057241

§ 371 Date: Apr. 17, 2019

Related U.S. Application Data
(60) Provisional application No. 62/409,721, filed on Oct. 18, 2016.

(51) Int. Cl. A61K 31/704; A61K 47/42; A61K 9/00; A61P 35/00

(52) U.S. Cl. CPC A61K 31/704; A61P 35/00; A61K 9/0024; A61K 47/42

(57) ABSTRACT

A chemotherapeutic-releasing implantable stick is described herein. The chemotherapeutic-releasing implantable stick includes an implantable stick having a length and thickness to fit within a needle track from a needle biopsy, the implantable stick providing a biocompatible and biodegradable substrate for the release of a chemotherapeutic agent when implanted. The chemotherapeutic-releasing implantable stick further includes a chemotherapeutic agent absorbed into the implantable stick.

### CROSS-REFERENCE TO RELATED APPLICATIONS

This application claims priority benefit from U.S. Provisional Patent Application No. 62/409,721 filed on Oct. 18, 2016, the entire content of which is incorporated herein by reference. All references cited anywhere in this specification, including the Background and Detailed Description sections, are incorporated by reference as if each had been individually incorporated.

### BACKGROUND

### 1. Technical Field

The field of the currently claimed embodiments of the present disclosure relates to chemotherapeutic-releasing implantable sticks, methods of producing chemotherapeutic-releasing implantable sticks, and precursor materials for chemotherapeutic-releasing implantable sticks.

### 2. Discussion of Related Art

Percutaneous needle biopsy is widely practiced for diagnosis of various cancers, including breast, kidney, liver, head and neck, thyroid, lung, pancreatic cancer and melanoma. In the majority of cases, a biopsy is performed to confirm a putative diagnosis of malignancy. Moreover, with the advent of personalized medicine, obtaining tumor tissue has gained even more importance to optimize treatment decisions.

Various needle devices are currently used and the two main types of biopsies are fine needle aspiration biopsy (FNAB) and core needle biopsy [1, 2]. FNAB utilizes a small-caliber needle, commonly from 22 G to 25 G, to remove tumor cells by aspiration without preserving the histological architecture of the tissue. A core needle biopsy is performed with a larger hollow needle to withdraw small cylinders of tissue from the suspected tumor. A biopsy needle with an outer sheath (TruCut) is often used in the procedure.

With either biopsy approach, cancer cells that are in general less adherent can detach from the tumor and colonize the surrounding tissue and beyond [3]. Metastasis and/or local invasion initiated by the biopsy procedure can occur in various ways—when the dislodged cancer cells enter the blood or lymphatic circulation, or loose cells left in the needle track by the retracting needle or displaced cells move with fluid pressure up the needle track [4].

The frequency and significance of tumor seeding associated with needle biopsies in various cancers remain largely controversial in spite of numerous surveys and studies. A number of early studies may have greatly underestimated the seeding rate as they were based on patient and physician reporting, without verification by an active cross-sectional imaging or histological analysis[5]. Biopsies of breast cancer appeared to be most prone to needle track tumor seeding, with up to 22% of the patients affected in seven studies in which needle tracks underwent histological analysis following surgical excision shortly after the biopsy [4]. It is important to note that not all tumor cells initially seeded along the needle track will result in metastasis because dislodged tumor cells will have to escape immune surveillance and other defense mechanisms in order to assure survival and local expansion. In addition, tumor seeding have been reported with percutaneous needle biopsies in lung, liver, renal, head and neck cancers but with a lesser frequencies [3, 4].

Likewise, in preclinical animal studies tumor seeding also poses a significant challenge, mainly resulted from the tumor implantation procedure itself. Excessive tumor seeding into the surrounding can potentially distort the results of therapeutic studies and affect the reproducibility in unpredictable ways. One illustrative example is the intracranial implantation of brain tumor cells in rodents, which has been a very useful tool for studying brain tumor biology and therapeutic development [6]. Blood-brain barrier (BBB) is a critical limitation restricting the majority of cancer therapeutics from reaching the brain tumor [7]. We observed that in intracranial rodent brain tumor models, malignant tumors often grow along the needle track up to the burr hole and fuse with the meninges. This growth along the needle tract altered the response to certain therapies, and created a different microenvironment that often resulted in a more aggressive growth pattern, shorter survival and potentially changed local BBB status.

Therefore, there remains a need for improved devices and approaches to help prevent tumor seeding during needle biopsy.

### SUMMARY OF THE DISCLOSURE

An aspect of the present disclosure is to provide a chemotherapeutic-releasing implantable stick. The chemotherapeutic-releasing implantable stick includes an implantable stick having a length and thickness to fit within a needle track from a needle biopsy, the implantable stick providing a biocompatible and biodegradable substrate for the release of a chemotherapeutic agent when implanted. The chemotherapeutic-releasing implantable stick further includes a chemotherapeutic agent absorbed into the implantable stick.

Another aspect of the present disclosure is to provide a method of producing a chemotherapeutic-releasing implantable stick. The method includes forming a layer of a biocompatible and biodegradable material, and applying a measured quantity of chemotherapeutic agent to the layer of biocompatible and biodegradable material such that the measured quantity of the chemotherapeutic agent is absorbed into the layer of biocompatible and biodegradable material. The method further includes rolling the layer of biocompatible and biodegradable material to form the chemotherapeutic-releasing implantable stick.

A further aspect of the present disclosure is to provide a precursor for producing a chemotherapeutic-releasing implantable stick. The precursor includes a gelatin powder; and a measured ratio of chemotherapeutic agent to gelatin powder absorbed into the gelatin powder such that formation of the chemotherapeutic-releasing implantable stick with a preselected length and thickness will have a predetermined quantity of the chemotherapeutic agent.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1A shows in a top most image of a gelatin stick supplemented with doxorubicin (DXR) for inserting in subcutaneous applications, with the gelatin stick being made by soaking and rolling a 20×3×1 mm Gelform® sponge in doxorubicin solution and left drying overnight, the lower images correspond to needles used for inserting the gelatin-doxorubicin stick (GDS), such as a 16 gauge needle with sheath, according to an embodiment of the present disclosure;

FIG. 1B shows a GDS inserted subcutaneously in an athymic nude mouse, according to an embodiment of the present disclosure;

FIG. 1C shows GDSs made by 100 μg/m1 (light upper arrowhead) or 500 μg/ml (dark lower arrowhead) doxorubicin solution were implanted subcutaneously in an athymic nude mouse and evaluated for over a month for skin appearance, wherein both GDSs were absorbed after 31 days without obvious skin damage in an experiment done in five mice, according to an embodiment of the present disclosure.

FIG. 2A illustrates a simulation of core needle biopsy performed with a 16 G needle with sheath into a subcutaneous SKMEL2 tumor on the left side of the mouse using a 16 G needle with sheath and a GDS was implanted in the needle track (a similar procedure was done to tumor on the right side without GDS implantation), upper and lower arrowheads indicate subcutaneous SKMEL2 tumors, GDS prevented tumor seeding in biopsy of subcutaneous SKMEL2 tumor, according to an embodiment of the present disclosure;

FIG. 2B shows an example of extensive bleeding (upper arrowhead) during the core needle biopsy of SKMEL2 tumor, according to an embodiment of the present disclosure;

FIG. 2C shows image scans for tumor seeding monitored by luciferase activity via Xenogen, and luceferase signal for right side (no GDS) and left side (with GDS), ten days after the biopsy in two mice as shown, according to an embodiment of the present disclosure;

FIG. 2D shows transverse sections of subcutaneous GDS and seeding tumor resulted from needle biopsy, SKMEL2 human melanoma cells expressing luciferase were grown subcutaneously in athymic nude mice and biopsy was performed by inserting a 16 G needle with sheath subcutaneously as shown in FIG. 1B, according to an embodiment of the present disclosure;

FIG. 3A shows image scans of GL261 mouse glioma cells expressing luciferase grown subcutaneously in C57BL6 mice and a simulation of core needle biopsy performed by inserting a 16 G needle with sheath subcutaneously as shown in FIG. 2A, a GDS was implanted in the needle track on the left side and no GDS was implanted to the right side as control, ten days after the biopsy, tumor seeding was monitored by luciferase activity via Xenogen, according to an embodiment of the present disclosure;

FIG. 3B is a plot of luciferase signal where no GDS is implanted in a right side and a GDS is implanted on left side of a mouse, according to an embodiment of the present disclosure;

FIG. 3C shows transverse sections of subcutaneous GDS and seeding tumor resulting from needle biopsy, GDS prevented tumor seeding in biopsy of subcutaneous GL261 tumor, according to an embodiment of the present disclosure;

FIG. 4A shows a survival rate in a F98 glioma model wherein F98 rat glioma cells implanted intra-cranially in F344 rats without (control) or with a 1-mm GDS, the survival curve of rats, a comparison showed marginal but insignificant difference (P=0.051), where m corresponds to the median survival in days after implantation, according to an embodiment of the present disclosure;

FIG. 4B are images showing a macroscopic appearance of rat brains implanted with F98 glioma without (−) or with (+) GDS, in the brain without GDS, tumor outgrew around the burr hole and was connected with meninges (as indicated by the arrowhead), according to an embodiment of the present disclosure; and

FIG. 4C show coronal image sections of rat brains implanted with F98 glioma without or with GDS (H&E staining), as shown on the right image, GDS prevented meningeal growth of brain tumor implantation, according to an embodiment of the present disclosure.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Needle biopsy is an indispensable diagnostic tool in obtaining tumor tissue for diagnostic examination. Tumor cell seeding in the needle track during percutaneous needle biopsies has been reported for various types of cancers. The mechanical force of the biopsy both directly displaces the malignant cells and causes bleeding and fluid movement that can further disseminate cells. To prevent the risk of tumor cell seeding during biopsy, we developed a gelatin stick loaded with chemotherapeutics such as doxorubicin (DXR) that was inserted into the biopsy canal. The gelatin-doxorubicin sticks (GDSs) were created by passively loading precut gelatin foam strips (Gelfoam) with doxorubicin solution. The dried GDSs were inserted into the needle track through the sheath during the needle biopsy and eventually self-absorbed. We showed that this procedure prevented tumor seeding during needle biopsies in two subcutaneous tumor models. In an alternative application, using GDSs in intracranial brain tumor implantation avoided the outgrowth of tumor from the rodent brain, which could otherwise potentially fuse the tumor with the meninges and distort the results in therapeutic studies in rodent brain tumor models.

Accordingly, some embodiments of this invention relate to chemotherapeutic-releasing implantable sticks, methods of producing chemotherapeutic-releasing implantable sticks, and precursor materials for chemotherapeutic-releasing implantable sticks. Some embodiments of the current invention are also directed to reducing the risk of tumor cell seeding of various preclinical animal models via development and insertion of chemotherapeutic-loaded gelatin sticks into the needle track.

The following describes some more details of the current invention by way of some examples. The general concepts of the current invention are not limited to only the particular embodiments.

### Creating Doxorubicin-Loaded Gelatin Sticks for Implantation

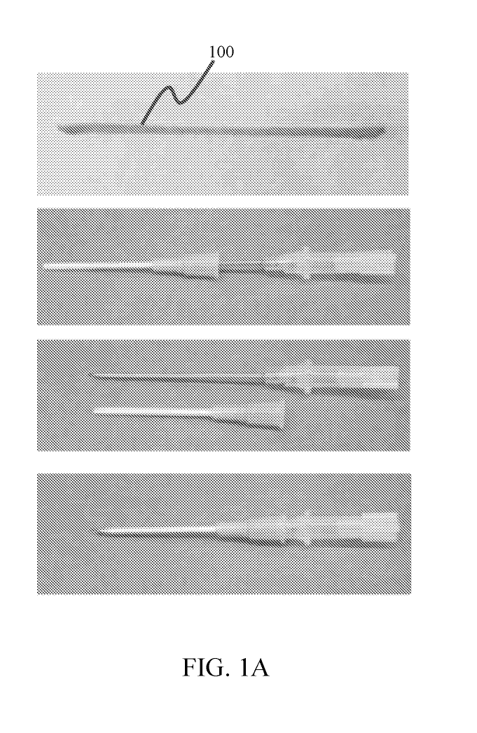

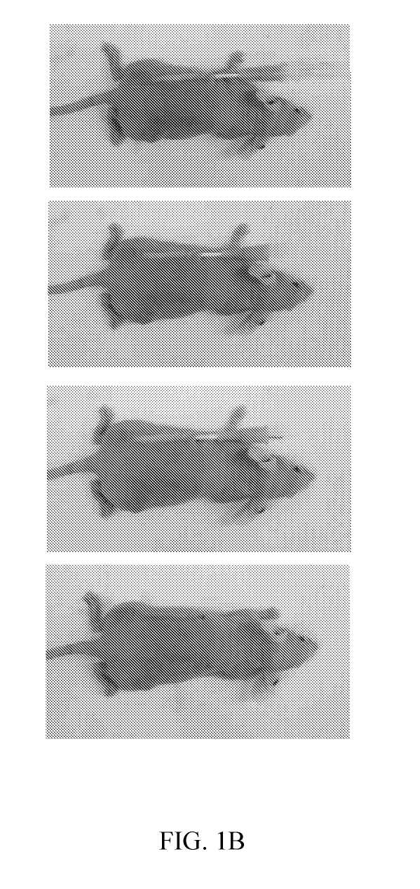

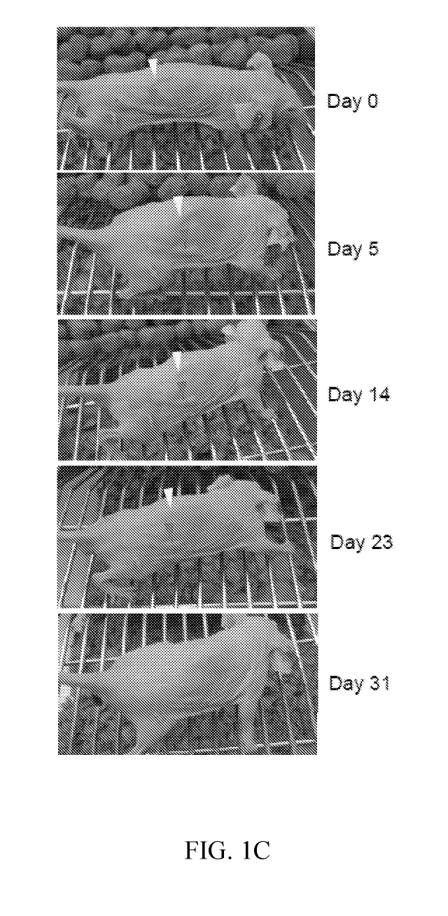

Gelfoam compressed sponge is a medical product intended as a hemostatic for bleeding surfaces during surgery. The commercially purchased gelfoams were cut into desired sizes, soaked in doxorubicin solutions, rolled and dried at 4° C. as described in the Materials and Methods to obtain dried GDSs. An image of a single dried GDS 100 is shown in the upper panel of FIG. 1A. Doxorubicin was chosen as a representative of chemotherapeutics because it demonstrated relatively low IC50 with the selected cancer cell lines compared to other commonly used chemotherapeutics including paclitaxel, topotecan, CPT-11, docetaxel, carboplatin and temozolomide, which have been tested in our previous study [8]. The following is a chemical formula of Doxorubicin:

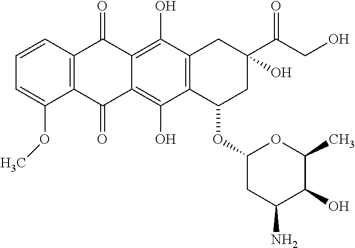 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

After saturating with doxorubicin of various concentrations, the dried GDSs 100 were 20-22 mm long and 1 mm wide (see, FIG. 1A upper panel), and were stored at −20° C. GDSs were rigid and could pass a sheath of a 16 G needle. In a subcutaneous implantation of GDS, a 16 G needle with a sheath was first inserted under the skin of an athymic nude mouse that carried the respective subcutaneous tumor. After tissue collection and withdrawal of the needle, a GDS was inserted through the sheath and pushed to the end by the needle while the sheath was retracted, with the GDS remaining in the skin (FIG. 1B). To evaluate the potential subcutaneous toxicity, two different CDSs, made of 250 (yellow arrowhead) or 500 μg/ml (red arrowhead) doxorubicin, were implanted in athymic nude mice (FIG. 1B). Over a course of a month, no subcutaneous toxicity was observed in a group of five mice and the skin irregularity caused by the initial GDS implantation gradually disappeared.

### GDSs Prevented Tumor Seeding in Needle Biopsy of Tumors in Mice

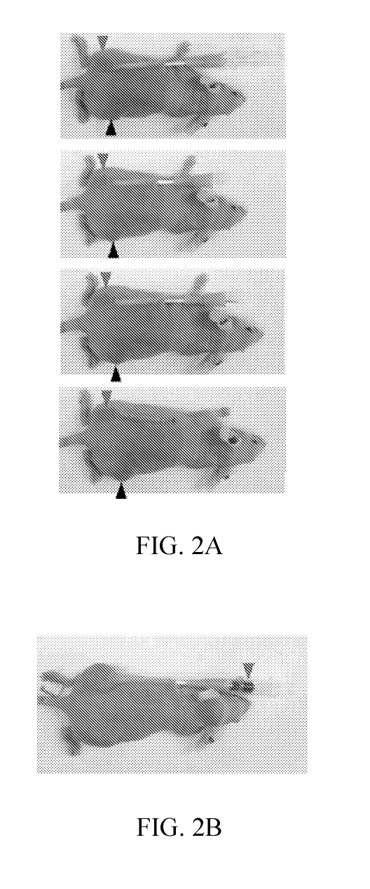

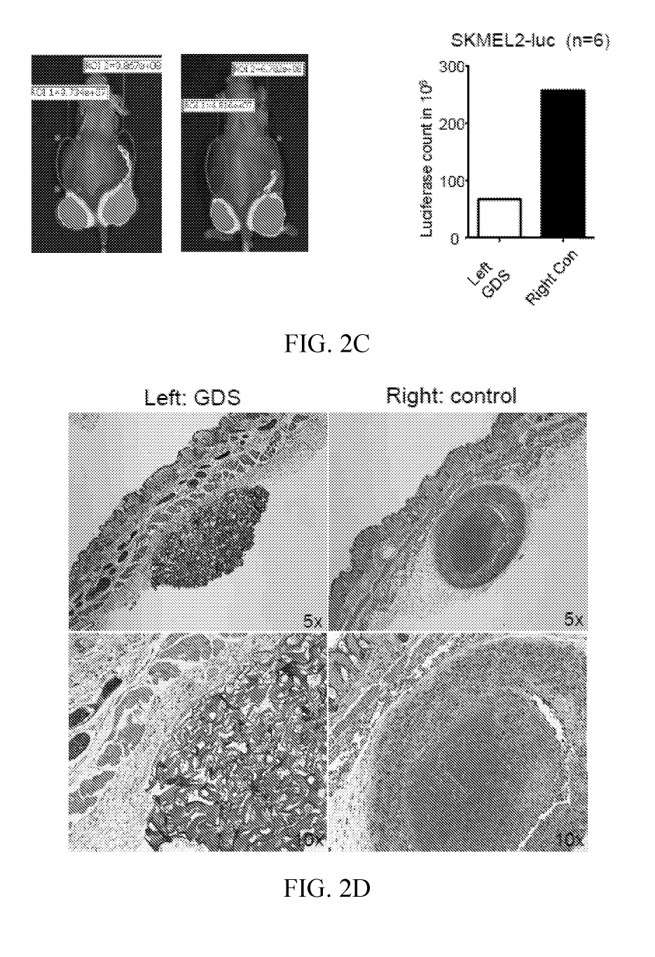

Tumor cell seeding after percutaneous needle biopsies can occur in various types of cancers [5, 9, 10]. In this study, we studied subcutaneously grown SKMEL2, a highly aggressive human melanoma xenograft, to test tumor seeding after core needle biopsy and the efficacy of GDS. We used a 16 G needle with a coaxial sheath to simulate the coaxial core needle biopsy. SKMEL2 cells were transfected with luciferase to monitor tumor growth. A 16 G needle with sheath was inserted underneath the skin, running 20-25 mm before penetrating into the tumor core. After retraction of the needle, a GDS saturated with 250 μg/ml doxorubicin was implanted (FIG. 1A). Penetration of the needle into the tumor caused tumor bleeding, with the blood running along the sheath to subsequently fill the needle track. FIG. 1B illustrates an occurrence of this extensive bleeding. Both the tip of the sheath and the blood from tumor vasculatures potentially become sources of tumor cell seeding in the needle track. Ten day after the biopsy, tumor cell seeding was evaluated by Xenogen. An evaluation of the untreated controls revealed tumor cell seeding along the needle track, while the needle track implanted with GDS remained largely tumor free (FIG. 2C). Subsequently, mice were euthanized and transverse sections of the skin samples showed the microscopic appearance of subcutaneous GDS and the tumor formation in the needle track (FIG. 2D).

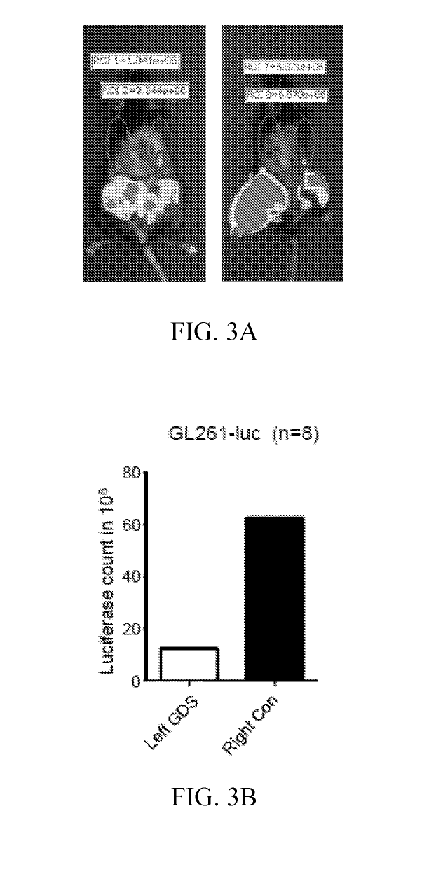

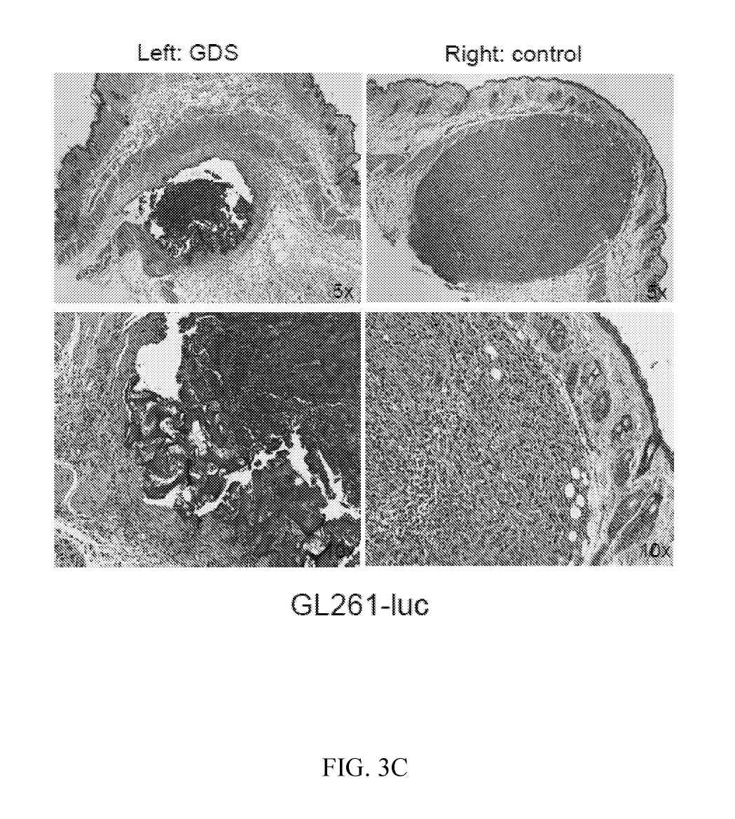

The same procedure was performed in C57BL6 mice implanted subcutaneously with syngeneic GL261 glioma cells. Similarly, ten days after the needle biopsy, the control side showed increased incidents of tumor seeding in the needle tracks, as reflected by the luciferase signals, in comparison to the contralateral side implanted with GDS (FIGS. 3A and 3B). H&E staining of the skin sections confirmed the tumor growth in the needle track on the control side (FIG. 3C, right panels).

### Using GDSs in Intracranial Implantation of Brain Tumor Cells

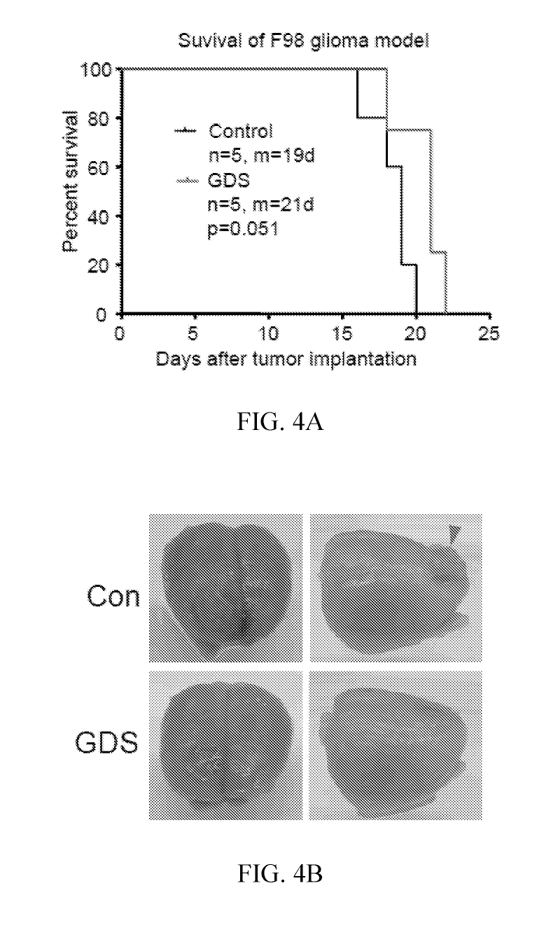

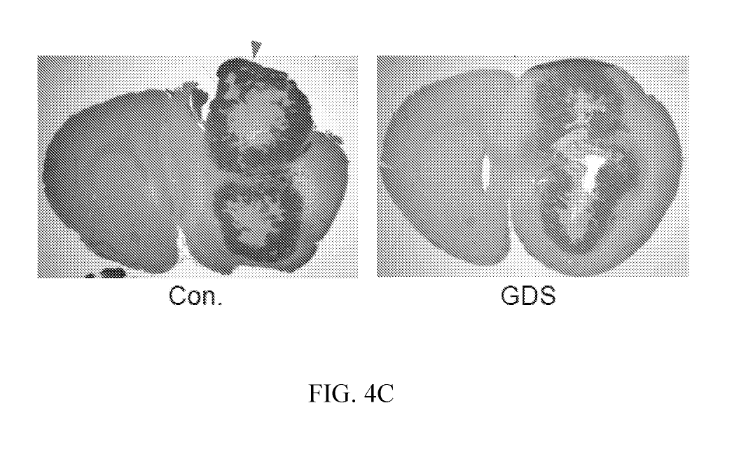

Intracranial implantation in rodents is a highly useful tool in brain tumor research and therapeutic development. As the blood-brain barrier (BBB) poses a critical restriction for drug delivery in the brain tumor, rodent brain tumor models should reflect this predicament. Yet, it has been observed that certain implanted brain tumors could grow along the needle track towards the burr hole and end up fusing with the meninges that are not restricted by the BBB. This could distort the results of therapeutic assessment in brain tumor models. Similar to the seeding of tumor cells in needle biopsy, aside from the risk of dragging tumor cells along the needle track during retraction of the implantation needle, bleeding caused by the needle could fill the needle track with tumor cells-containing blood all the way to the meninges and burr hole. In this study, we examined GDS in preventing this outgrowth of implanted brain tumors. Inserting a GDS as short as 1 mm into the burr hole without penetrating into the brain tissue did not significantly alter the survival of syngeneic F98 rat glioma model (FIG. 4A). In the control group, the fully grown F98 tumor protruded outside the brain surface (FIGS. 4B and 4C) and grew adjacent or possibly attached to the meninges. In contrast, the brain tumor implanted with GDS did not spread out of the brain surface to the meninges even up to the point where the tumor resulted in the animal's death (FIGS. 4B and 4C).

### Discussion

Gelatin is a mixture of proteins and peptides obtained by partial hydrolysis of collagen from the cartilage, skin and bones of animals. Gelatin products are safe for human consumption and medical applications. For example, Gelfoam compressed sponge is produced as a hemostatic for bleeding surfaces. Our choice of Gelfoam was based on its ease of handling and loading of chemotherapeutics. Gelatin is most commonly available as powder, which can be dissolved in aqueous solutions by heating and is hardened upon cooling and drying. It is also conceivable to produce GDS directly from the gelatin powder and doxorubicin solution using heating, cooling and drying. However, this will require more biomaterial development to produce standardized GDS fitting to the sheath of a biopsy needle of 16 G or thinner. In this study, we have shown the doxorubicin-loaded gelatin sticks prevented tumor seeding in needle tracks in two mouse tumor models, while being safe to use and causing no skin irritation in the usually sensitive athymic nude mice.

In order to minimize the risk of tumor seeding, several improvements have been introduced in the clinical practice including cryoablation and coaxial cutting needle technique. Percutaneous cryoablation guided by imaging is a minimally invasive biopsy procedure with a lower risk of needle-track seeding, which involves a two-step freezing method to kill tissue around the biopsy-needle sheath to avoid needle-track seeding [11, 12]. Coaxial cutting needle technique is used in the core needle biopsy that applies a needle introducer that remains in position during multiple cutting needle sampling, which may protect the needle track from tumor seeding [3]. In this study, we used the needle with a sheath that simulated the coaxial cutting needle technique. Despite the protection of the needle introducer/sheath, excessive bleeding caused by penetrating and sampling the tumor tissues can fill the blood into the needle track and potentially seed dislodged tumor cells. Also detached tumor cells attached to the sheath can be left behind in the needle track. We further demonstrated that implanting GDS could prevent such seeding in the rodent flank tumor models. It is noteworthy that percutaneous biopsies of organs such as kidney, lung and liver entail penetration of internal organs and body cavities that can trap loose tumor cells outside the needle tracks. Thus, it is feasible that a combination of cryoablation, coaxial cutting needle and GDS implantation could minimize the risk of tumor seeding.

By performing intracranial brain tumor implantation in rodents, we observed the outgrowth of tumor out of brain surface at the burr hole occurred in certain brain tumors. For example, the syngeneic GL261 glioma in C57BL6 mice usually does not form outgrown tumor fused to the meninges. It is noted that the application of GDS in mouse models is less feasible due to small size of the mouse skull. In contrast, syngeneic F98 and 9L rat gliomas can often bulge out of the brain surface and potentially fuse with the meninges, in which GDS would be a useful device. We also note that the GDS implantation is useful to prevent tumor outgrowth along the needle track that occurs during intracranial implantation of VX2 tumor cells in New Zealand white rabbits.

### Materials and Methods

### Cell Lines and Tissue Culture

The human melanoma cell line SKMEL2 was obtained from ATCC. Mouse glioma cell line GL261 expressing luciferase was described before [13]. All cells were maintained in DMEM media supplemented with 10% fetal bovine serum and antibiotics. Cells were kept in frozen stocks upon reception and were not additionally authenticated. Tissue culture was maintained at 37° C. in humidified air containing 5% CO2.

### Luciferase Expression by Lentivirus

Lunciferase expression was previously described [14]. Briefly, Firefly luciferase cDNA from pGL3-basic (Promega, Madison, Wis.) was subcloned in pFUGW and transfected along with CMVΔR8.91 and pMD.G in 293T cells by Lipofectamine 2000 (Invitrogen, Carlsbad, Calif.). Virus was harvested after 48 hours and SMKEL2 cells were infected by incubating with 8 μg/ml polybrene (Sigma, St. Louis, Mo.) in the growth medium.

### Animal Experiments

All animal works were approved by the Animal Care and Use Committee (ACUC) of the Johns Hopkins University.

Female athymic nude mice or C57BL6 mice, 5-6 weeks of age, were purchased from National Cancer Institute (Frederick, Md.). For the implantation procedure, female athymic nude mice were anesthetized via intraperitoneal injection of 60 μl of a stock solution containing ketamine hydrochloride (75 mg/kg) (100 mg/mL; Ketamine HCl; Abbot Laboratories, Chicago, Ill., USA) and xylazine (7.5 mg/kg) (100 mg/mL; Xyla-ject®; Phoenix Pharmaceutical, St. Joseph, Mo., USA) in a sterile 0.9% NaCl solution. 5×10⁶ GL261 or SKMEL2 cells in 100 μl expressing luciferase were mixed with equal volume of Matrigel (BD Bioscience) and injected subcutaneously in the flanks of mice.

Luciferase activity was determined by a Xenogen instrument (IVIS 200) with intraperitoneal injection of 2 mg/mouse D-luciferin potassium salt solution (Gold Biotechnology, St. Louis, Mo.). After 15 min following the injection, the animals were scanned for 1 min at a distance of 20 cm.

### Making and Application of Gelatin-Doxorubicin Sticks (GDSs)

Gelfoam absorbable gelatin sponges (2×2 cm) were manufactured by Pfizer. A piece of 20×4×1 mm was cut out from the sponge by a scalpel and soaked in doxorubicin hydrochloride (DXR) solution for two minutes. The gelatin piece was then rolled in the form of a stick, straightened, air-dried on a Petri dish over night at 4° C. and stored at −20° C.

Mice were anesthetized first and a 16 G catheter needle (Jelco, No. 4042) was inserted under the skin for about 20 mm. For mice with subcutaneous tumors, the needle penetrated into the tumor body and rotated a round in order to displace sufficient amount of tumor cells. The needle was then retracted out of the skin with the sheath being left inside. At this point, a 20 mm GDS was inserted through the sheath by a fresh 16 G needle and left inside after removal of the sheath. For the tumor on the control side, no GDS was inserted.

For brain tumor implantation in rats, female F344 Fisher rats (weight 100-150 gram) were purchased from the NCI. Rats were anesthetized via intraperitoneal (i.p) injection composed of ketamine hydrochloride (75 mg/kg; 100 mg/mL; ketamine HCl; Abbot Laboratories) and xylazine (7.5 mg/kg; 100 mg/mL; Xyla-ject; Phoenix Pharmaceutical) in a sterile 0.9% NaCl solution. Subsequently, rat F98 glioma cells at 20,000 cells/μ1 were loaded in a 24 G Hamilton syringe needle (7105KH) and the needle tip was cleaned from tumor cells with an ethanol wipe. The needle was inserted stereotactically into the burr hole located 3 mm lateral and 2 mm anterior to the bregma in the depth of 6 mm. After a 1 min pause, the needle was retracted by 1 mm and 1 μl of cells were injected slowly over 1 min. After pausing for 5 min, the needle was slowly removed. For the implantation with GDS, a piece of 1 mm GDS created with 50 μg/ml doxorubicin was inserted in the burr hole. No GDS was used in the control rats. The burr hole was sealed by bone wax and the skull was irrigated by 0.5 ml sterile PBS.

As it can be appreciated from the above paragraphs, there is provided a chemotherapeutic-releasing implantable stick. The chemotherapeutic-releasing implantable stick includes an implantable stick having a length and thickness to fit within a needle track from a needle biopsy, the implantable stick providing a biocompatible and biodegradable substrate for the release of a chemotherapeutic agent when implanted, as shown for example in FIGS. 1A and 1B. The chemotherapeutic-releasing implantable stick further includes a chemotherapeutic agent absorbed into the implantable stick.

In an embodiment, the implantable stick has a thickness corresponding to a needle gauge of 7 to a needle gauge of 34. In an embodiment, the implantable stick consists essentially of gelatin. In an embodiment, the chemotherapeutic agent is an anticancer agent. In an embodiment, the chemotherapeutic agent comprises at least one of doxorubicin, paclitaxel, topotecan, CPT-11, docetaxel, carboplatin and temozolomide. In an embodiment, the chemotherapeutic agent comprises doxorubicin. In an embodiment, the chemotherapeutic agent consists essentially of doxorubicin.

As it can be appreciated from the above paragraphs, there is also provided a method of producing a chemotherapeutic-releasing implantable stick. The method includes forming a layer of a biocompatible and biodegradable material, and applying a measured quantity of chemotherapeutic agent to the layer of biocompatible and biodegradable material such that the measured quantity of the chemotherapeutic agent is absorbed into the layer of biocompatible and biodegradable material. The method further includes rolling the layer of biocompatible and biodegradable material to form the chemotherapeutic-releasing implantable stick.

As it can be further appreciated from the above paragraphs, there is further provided a precursor for producing a chemotherapeutic-releasing implantable stick. The precursor includes a gelatin powder; and a measured ratio of chemotherapeutic agent to gelatin powder absorbed into the gelatin powder such that formation of the chemotherapeutic-releasing implantable stick with a preselected length and thickness will have a predetermined quantity of the chemotherapeutic agent.

In an embodiment, the chemotherapeutic agent is an anticancer agent. In an embodiment, the chemotherapeutic agent comprises at least one of doxorubicin, paclitaxel, topotecan, CPT-11, docetaxel, carboplatin and temozolomide. In an embodiment, the chemotherapeutic agent comprises doxorubicin. In an embodiment, the chemotherapeutic agent consists essentially of doxorubicin.

### REFERENCES

1. Cao H, Kao R H, Hsieh M C. Comparison of core-needle biopsy and fine-needle aspiration in screening for thyroid malignancy: a systematic review and meta-analysis. Curr Med Res Opin. 2016: 1-11. doi:
2. Ocak S, Duplaquet F, Jamart J, Pirard L, Weynand B, Delos M, Eucher P, Rondelet B, Dupont M, Delaunois L, Sibille Y, Dahlqvist C. Diagnostic Accuracy and Safety of CT-Guided Percutaneous Transthoracic Needle Biopsies: 14-Gauge versus 22-Gauge Needles. J Vasc Intery Radiol. 2016; 27: 674-81. doi:
3. Shyamala K, Girish H C, Murgod S. Risk of tumor cell seeding through biopsy and aspiration cytology. J Int Soc Prey Community Dent. 2014; 4: 5-11. doi:
4. Robertson E G, Baxter G. Tumour seeding following percutaneous needle biopsy: the real story! Clin Radiol. 2011; 66: 1007-14. doi:
5. Chen I, Lorentzen T, Linnemann D, Nolsoe C P, Skjoldbye B, Jensen B V, Nielsen D. Seeding after ultrasound-guided percutaneous biopsy of liver metastases in patients with colorectal or breast cancer. Acta Oncol. 2015: 1-6. doi:
6. Bai R Y, Staedtke V, Riggins G J. Molecular targeting of glioblastoma: Drug discovery and therapies. Trends Mol Med. 2011; 17: 301-12. doi:
7. Chico L K, Van Eldik L J, Watterson D M. Targeting protein kinases in central nervous system disorders. Nat Rev Drug Discov. 2009; 8: 892-909. doi:
8. Staedtke V, Bai R Y, Sun W, Huang J, Kibler K K, Tyler B M, Gallia G L, Kinzler K, Vogelstein B, Zhou S, Riggins G J. Clostridium novyi-NT can cause regression of orthotopically implanted glioblastomas in rats. Oncotarget. 2015; 6: 5536-46. doi:
9. Viswanathan A, Ingimarsson J P, Seigne J D, Schned A R. A single-centre experience with tumour tract seeding associated with needle manipulation of renal cell carcinomas. Can Urol Assoc J. 2015; 9: E890-3. doi:
10. Valle L G, Rocha R D, Mendes G F, Succi J E, Andrade J R. Tumor seeding along the needle track after percutaneous lung biopsy. J Bras Pneumol. 2016; 42: 71. doi:
11. Yamauchi Y, Izumi Y, Kawamura M, Nakatsuka S, Yashiro H, Tsukada N, Inoue M, Asakura K, Nomori H. Percutaneous cryoablation of pulmonary metastases from colorectal cancer. PLoS One. 2011; 6: e27086. doi:
12. Mu F, Liu S P, Zhou X L, Chen J B, Li H B, Zuo J S, Xu K C. Prevention of needle-tract seeding by two-step freezing after lung cancer biopsy. Pathol Oncol Res. 2013; 19: 447-50. doi:
13. Bai R Y, Staedtke V, Aprhys C M, Gallia G L, Riggins G J. Antiparasitic mebendazole shows survival benefit in 2 preclinical models of glioblastoma multiforme. Neuro Oncol. 2011; 13: 974-82. doi:
14. Bai R Y, Staedtke V, Wanjiku T, Rudek M A, Joshi A D, Gallia G L, Riggins G J. Brain Penetration and Efficacy of Different Mebendazole Polymorphs in a Mouse Brain Tumor Model. Clin Cancer Res. 2015. doi:

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A chemotherapeutic-releasing implantable stick, comprising:
an implantable stick having a length and thickness to fit within a needle track from a needle biopsy, said implantable stick providing a biocompatible and biodegradable substrate for the release of a chemotherapeutic agent when implanted; and
a chemotherapeutic agent absorbed into said implantable stick.

2. The chemotherapeutic-releasing implantable stick according to claim 1, wherein said implantable stick has a thickness corresponding to a needle gauge of 7 to a needle gauge of 34.

3. The chemotherapeutic-releasing implantable stick according to claim 1, wherein said implantable stick consists essentially of gelatin.

4. The chemotherapeutic-releasing implantable stick according to claim 1, wherein said chemotherapeutic agent is an anticancer agent.

5. The chemotherapeutic-releasing implantable stick according to claim 1, wherein said chemotherapeutic agent comprises at least one of doxorubicin, paclitaxel, topotecan, CPT-11, docetaxel, carboplatin and temozolomide.

6. The chemotherapeutic-releasing implantable stick according to claim 1, wherein said chemotherapeutic agent comprises doxorubicin.

7. The chemotherapeutic-releasing implantable stick according to claim 6, wherein said chemotherapeutic agent consists essentially of doxorubicin.

8. A method of producing a chemotherapeutic-releasing implantable stick, comprising:
forming a layer of a biocompatible and biodegradable material;
applying a measured quantity of chemotherapeutic agent to said layer of biocompatible and biodegradable material such that said measured quantity of said chemotherapeutic agent is absorbed into said layer of biocompatible and biodegradable material; and
rolling said layer of biocompatible and biodegradable material to form said chemotherapeutic-releasing implantable stick.

9. The method according to claim 8, wherein said layer of biocompatible and biodegradable material consists essentially of gelatin.

10. The method according to claim 8, wherein said chemotherapeutic agent is an anticancer agent.

11. The method according to claim 8, wherein said chemotherapeutic agent comprises at least one of doxorubicin, paclitaxel, topotecan, CPT-11, docetaxel, carboplatin and temozolomide.

12. The method according to claim 8, wherein said chemotherapeutic agent comprises doxorubicin.

13. The method according to claim 12, wherein said chemotherapeutic agent consists essentially of doxorubicin.

14. A precursor for producing a chemotherapeutic-releasing implantable stick, comprising:
a gelatin powder; and
a measured ratio of chemotherapeutic agent to gelatin powder absorbed into said gelatin powder such that formation of said chemotherapeutic-releasing implantable stick with a preselected length and thickness will have a predetermined quantity of said chemotherapeutic agent.

15. The precursor according to claim 14, wherein said chemotherapeutic agent is an anticancer agent.

16. The precursor according to claim 14, wherein said chemotherapeutic agent comprises at least one of doxorubicin, paclitaxel, topotecan, CPT-11, docetaxel, carboplatin and temozolomide.

17. The precursor according to claim 14, wherein said chemotherapeutic agent comprises doxorubicin.

18. The precursor according to claim 12, wherein said chemotherapeutic agent consists essentially of doxorubicin.
